(19) United States
(12) Patent Application Publication
(10) Pub. No.: US 2008/0171857 A1
(43) Pub. Date: Jul. 17, 2008

(54) Process for the Purification of Recombinant Granulocyte-Colony Stimulating Factor

(76) Inventors: Uma Devi Komath, Andhra Pradesh (IN); Anupama Nandamuri, Andhra Pradesh (IN); Ashok Kumar Nuvvula, Andhra Pradesh (IN); Srilalitha Movva, Andhra Pradesh (IN); Sreenivasu Karra, Andhra Pradesh (IN); Mitali Samaddar, Andhra Pradesh (IN); Jayaram Chigurupati, Andhra Pradesh (IN)

Correspondence Address: VENABLE LLP, P.O. BOX 34385, WASHINGTON, DC, 20043-9998, US

(21) Appl. No.: 11/886,415

(22) PCT Filed: Mar. 13, 2006
(86) PCT No.: PCT/IN06/00090

§ 371 Date: Sep. 17, 2007

(30) Foreign Application Priority Data
Mar. 17, 2005 (IN) 274/CHE/2005

(51) Int. Cl. C07K 1/18

(52) U.S. Cl. 530/416

(57) ABSTRACT

The present invention describes a novel process for large scale purification of therapeutic grade quality of recombinant human G-CSF from microbial cells, wherein the protein is expressed as inclusion bodies. The process involves the novel use of Hydrophobic Interaction Chromatography (HIC) step to purify G-CSF eluted from a cation exchange column. A combination of these two chromatography steps provides good purity and yields which are essential for a production scale process. The host cell related contaminants like proteins, DNA and endotoxins are estimated to be within the specifications outlined by the drug regulatory authorities.

### FIELD OF THE INVENTION

The present invention relates to a novel process for die isolation of therapeutically pure and biologically active recombinant human granulocyte-colony stimulating factor (G-CSF) from inclusion bodies expressed in microbial cells. The process leads to the purification of biologically active G-CSF in high yields, free from its oligomeric forms and other host cell proteins. More specifically, the invention is directed to a process for the large-scale production of a therapeutically pure and biologically active G-CSF protein in solution by the use of hydrophobic interaction chromatography.

### BACKGROUND OF THE INVENTION

Human granulocyte-colony stimulating Factor belongs to a group of colony stimulating factors that play an important role in stimulating the differentiation and proliferation of hematopoietic precursor cells and activation of mature neutrophils. G-CSF is capable of supporting neutrophil proliferation in vitro and in vivo. Large quantities of recombinant G-CSF have been produced in genetically engineered Escherichia coli and have been successfully used in the clinic to treat cancer patients suffering from chemotherapy-induced neutropenia. E. coli produced G-CSF is a 175 amino acid polypeptide chain containing an extra methionine at its N-terminus. This protein has been produced by expressing a G-CSF gene in E. coli and purifying it to homogeneity.

Many earlier patents have described various aspects of recombinant expression and purification of the G-CSF protein from different expression systems ranging from bacterial cells to yeast and mammalian cells. Some of the processes described arc multi-step processes where losses in yield at the end of the purification process can be significant. A few other purification processes described in patent literature appear to be simpler but no mention is made of the therapeutic grade quality of the purified material. Hence, there is a need for a simplified procedure that is easily scaleable for production, gives higher yields and produces purified G-CSF protein of therapeutic grade quality. In an earlier patent for a process (WO 04001056A1) for G-CSF, we have addressed most of the limitations of lengthy processes described in scientific literature. In this patent we have tried to develop a novel process by the inclusion of a unique hydrophobic interaction chromatography step that has not been hitherto described for the purification of G-CSF. The G-CSF protein purified by the two-step chromatography process that includes a hydrophobic interaction chromatography step as described in this patent, is free from higher multimeric aggregates of G-CSF, other host cell-related proteins, DNA and endotoxins.

Purification method described in U.S. Pat. No. 5,532,341 (Karl Welte, Sloan Kettering Institute) describes the purification of pluripotent granulocyte-colony stimulating factor from conditioned media using a three step chromatography process involving DEAE, gel nitration and reverse phase columns. The protein purified by this method was biologically active and homogenous. The procedure however was applicable to the G-CSF protein that was secreted in cell culture supernatants and concentrated by ammonium sulphate precipitation. The suitability of this process for G-CSF solubilized and refolded from inclusion bodies has not been demonstrated.

In an international patent WO 03/051922 A1 assigned to Gaberc Porekar and Menart, the purification of G-CSF is described by the use of an immobilized metal affinity chromatography (IMAC) matrix. This step is coupled to cation exchange and gel filtration chromatography steps to get a biologically active and pure G-CSF protein in solution. Although simple, but it still incorporates a minimum of three chromatography steps for final purification and the final yields of the protein are not clearly evident.

A simplified process for purification of recombinant G-CSF is mentioned in U.S. Pat. No. 5,055,555 patent. The purification method described applies to G-CSF protein secreted into the medium when expressed in yeast or mammalian expression systems. The protein is partially purified on a cation exchanger and precipitated from pooled column fractions by using high concentrations of sodium chloride in the range of 1.5 to 2.5M. For G-CSF recovered from inclusion bodies in bacteria, sodium chloride precipitation of the protein increases the aggregation status of the protein and hence gelling it back into solution after that, is likely to be a cumbersome process. Besides, this process does not assure the therapeutic grade purity or the protein.

In the U.S. Pat. No. 5,849,883 assigned to Amgen Inc., the process describes recovering human and bovine G-CSF expressed as inclusion bodies from a microbial system. The inclusion bodies are stated to be solubilised with Sarkosyl and purified using CM-Sepharose, a cation exchange chromatography step. In U.S. Pat. No. 5,830,705 assigned to Amgen, a process for the purification of G-CSF from COS cells is described. Since G-CSF is not expressed as inclusion bodies the method for purifying the recombinant protein is in principle different from an E. coli expressed protein. The U.S. Pat. Nos. 5,714,581 and 5,681,720 on G-CSF describe the various deletion and substitution derivatives of the protein and methods for producing these derivatives using microorganisms. In earlier patents assigned to Amgen Inc., U.S. Pat. No. 4,810,643 and U.S. Pat. No. 4,999,291 methods for G-CSF extraction from inclusion bodies produced in E. coli cells are described. The protein in inclusion bodies is extracted with deoxycholate (DOC), solubilized with an ionic detergent (Sarkosyl) and refolded. The refolded protein is purified on a CM-column followed by a G-75 gel filtration chromatography step. The European Patent EP 0243153 (Immunex Corporation) describes molecular level modifications to the human G-CSF and related mutant cDNAs for increasing expression in microbial systems and processes for making the proteins using these systems. Purification of crude G-CSF produced in supernatants of HBT 563 cells is achieved by ammonium sulphate precipitation followed by chromatography on gel filtration and preparative reverse phase-HPLC columns. In EP 0215126 patent assigned to Chugai Seiyaku Kabushiki Kaisha, the G-CSF protein is purified on an Ultrogel AcA54 column followed by precipitation of non-GCSF proteins with 30% n-propanol. The supernatant of 30% n-propanol precipitation step is loaded onto a C-18 reverse phase column and eluted with 40% n-propanol to gel a purified protein preparation.

The patents EP 0169566, WO 8604506 and WO 8604605 assigned to Chugai Seiyaku Kabushiki Kaisha describes a novel CSF having the ability to promote differentiation and proliferation of bone marrow cells, the human gene encoding a polypeptide with G-CSF activity and a method for obtaining recombinant expression of the same. WO 8703689 and EP 0237545 are patents by Kirin-Amgen for G-CSF. The former one describes immunological procedures associated with the production of murine monoclonal antibodies for the detection of G-CSF in biological fluids and the latter presents polynucleotide sequences coding for the human G-CSF and their analogs. The European patents EP 0272703, EP 0459630 and EP 0256843 disclose amino acid modifications of G-CSF, their expression and biological activities. British patent 2213821 discusses the construction of a synthetic human G-CSF gene. Australian Patent AU-A-76380/91 reports the construction of various muteins of G-CSF and their comparative activities. The U.S. Pat. No. 5,580,755 and U.S. Pat. No. 5,582,823 illustrate DNA sequences that encode part or all of the polypeptide sequence of G-CSF and their characterization.

Scientific literature describes various methods to purify G-CSF expressed in bacterial, yeast or mammalian cells but the multi-step processes are meant for small-scale isolation of the protein for characterization purposes only. The processes may not be easily scaleable for production and is not likely to yield a protein of therapeutic grade purity.

The different purification protocols discussed in the above patent literature involves multiple chromatography steps chiefly ion-exchange followed by reverse phase or gel filtration chromatography step. Purification of G-CSF protein on a hydrophobic interaction chromatography column has not been described so far. Hydrophobic interaction chromatography (HIC) involves the use of high molarities of salt in the protein solution but at concentrations that are below their precipitation points. At these salt concentrations, certain ligands, which under normal salt conditions would not adsorb these proteins, become excellent adsorbents. The principle for protein adsorption to HIC is complementary to ion exchange and gel filtration chromatography methods. The use of high molarities of salt restricts its use to those proteins that can withstand high conductivities. Highly hydrophobic proteins like G-CSF are practically unstable at such conductivities and hence the use of hydrophobic interaction chromatography is generally ruled out as a purification option. Here, we describe the use of HIC step lo further purify G-CSF eluted from a cation exchange chromatography step. This novel combination of cation exchange and hydrophobic interaction chromatography steps has not been reported so far in patent literature. Besides being novel, the process also shows good recoveries and is easily scaleable for industrial production of therapeutic grade G-CSF.

### SUMMARY OF THE INVENTION

The present invention provides a method for large scale purification of therapeutic grade recombinant human G-CSF, said method comprising the steps of:
isolating inclusion bodies containing G-CSF from microbial cells
solubilizing said G-CSF protein from isolated inclusion bodies
refolding the said solubilized G-CSF protein to obtain active folded protein
subjecting the said refolded G-CSF protein to two step chromatography wherein the said refolded G-CSF protein is first subjected to cation exchange chromatography followed by hydrophobic interaction chromatography

to obtain purified therapeutic grade G-CSF protein

The said G-CSF isolated from inclusion bodies is solubilized in a concentration of urea or guanidinium hydrochloride at alkaline pH and refolded at an acidic pH for 6 to 16 hrs at room temperature.

The said refolded protein is bound to a sulphonate, carboxymethyl or sulphopropyl functional group containing chromatography matrices.

The ion exchange column is run in the pH range of 3.5 to 5.5 using buffers of citrate, phosphate or acetate salts in the molarity range of 5 mM to 50 mM.

The said protein bound to the cation exchange group is eluled by increasing the ionic strength of the buffer by the addition of chloride, citrate or sulphate salts in the pH range of 4.0 to 6.0.

The G-CSF containing protein solution eluted from the cation exchange column is purified using hydrophobic interaction chromatography on resins having butyl, oclyl or phenyl functional groups.

The said column is equilibrated with buffers in the pH range of 4.0 to 7.0 containing ammonium sulphate salts in the molarity range of 0.25 M to 1.0 M.

The said column is eluled by decreasing the concentration of ammonium sulphate salt in the buffer and by optionally increasing the concentration of ethanol from 2 to 20% for enhanced recoveries.

### BRIEF DESCRIPTION OF THE ACCOMPANYING DRAWINGS

The invention will now be described with reference to the accompanying drawings.

FIG. 1 is a restriction map of E. coli expression vector, which directs the expression of G-CSF.

FIG. 2 is the complete nucleotide sequence of G-CSF and the derived amino acid sequence.

FIG. 3 is the chromatography profile of G-CSF after ion exchange chromatography

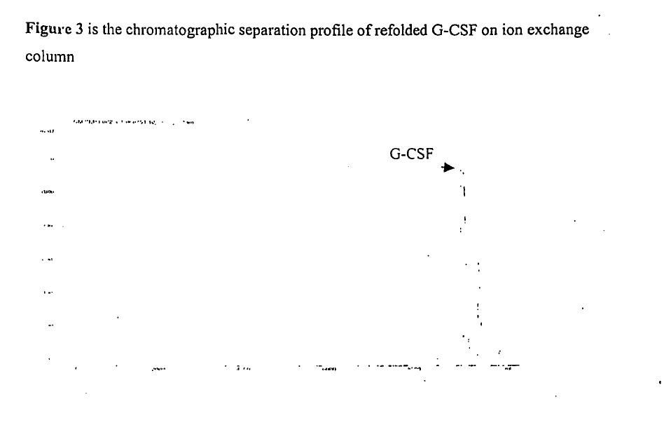

FIG. 4 is the chromatography profile of G-CSF after hydrophobic interaction chromatography

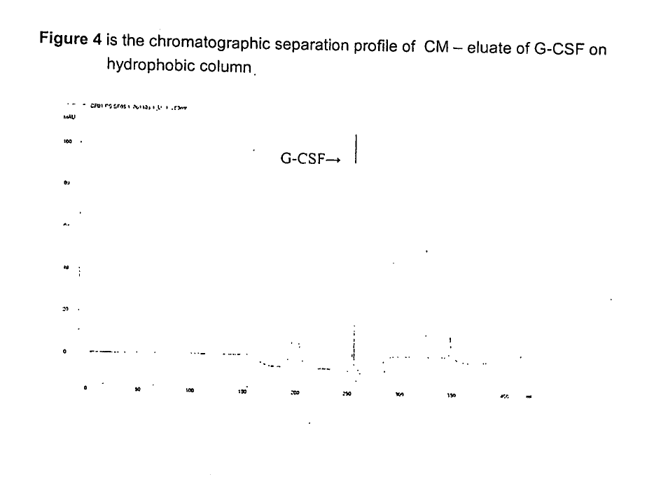

FIG. 5 is the SDS-PAGE profile of G-CSF after purification

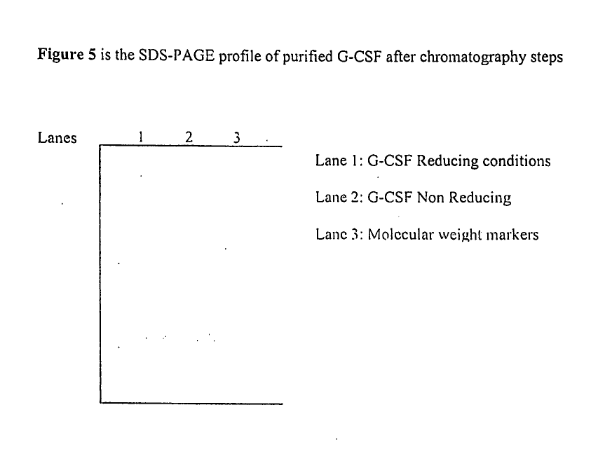

### DETAILED DESCRIPTION OF THE INVENTION

A simple and novel method involving a combination of ion exchange and hydrophobic interaction chromatography steps has been developed for large-scale purification of G-CSF solubilized from inclusion bodies expressed in microbial cells.

The G-CSF protein in this case is preferably produced by recombinant methods in bacterial expression systems. The G-CSF gene is isolated from a known source and ligated to a suitable expression vector, which is then used to transform an appropriate host strain. The recombinant microbial strain is grown by fermentation under suitable conditions that promote the maximum expression of the desired protein.

The isolation and purification process for G-CSF involves lysing the said cells by high-pressure homogenization or sonication and isolating the IB pellets by centrifugation. The G-CSF present in the IB is solubilized by using a chaotrope like urea or guanidinium chloride in the concentration range of 2.0 to 4.0 M and in a buffer of high pH. The protein is refolded at low pH, preferably in the range of 4.0 to 6.0 and the refolded protein is loaded on a cation exchange chromatography column at a low pH. Increasing the salt concentration in the buffer effects the elution of the protein and further purification is attempted by a hydrophobic chromatography step. In a preferred embodiment of this invention, although G-CSF is to a large extent purified by using a single ion-exchange chromatography step, but a combination of ion exchange with hydrophobic column ensures lot-to-lot reproducibility with feed streams that can have minor alterations when carried out at industrial scale.

The process described in the present invention can be applied for industrial scale purification of recombinant G-CSF to homogeneity and of therapeutic grade quality. The purified G-CSF protein has similar physico-chemical characteristics as the native protein.

### Cloning and Expression

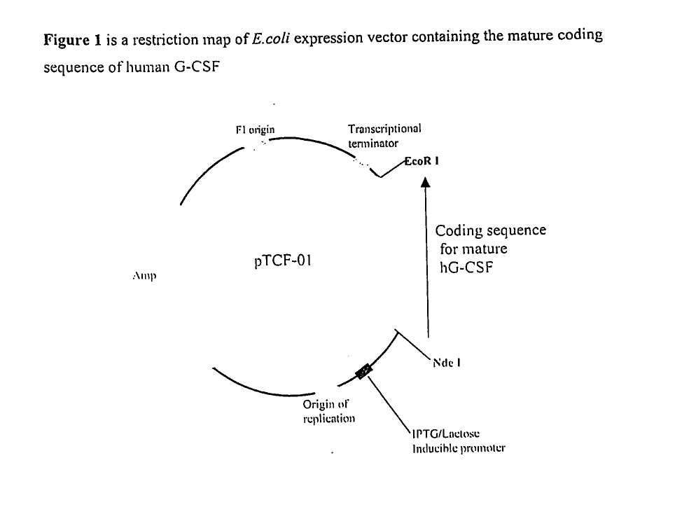

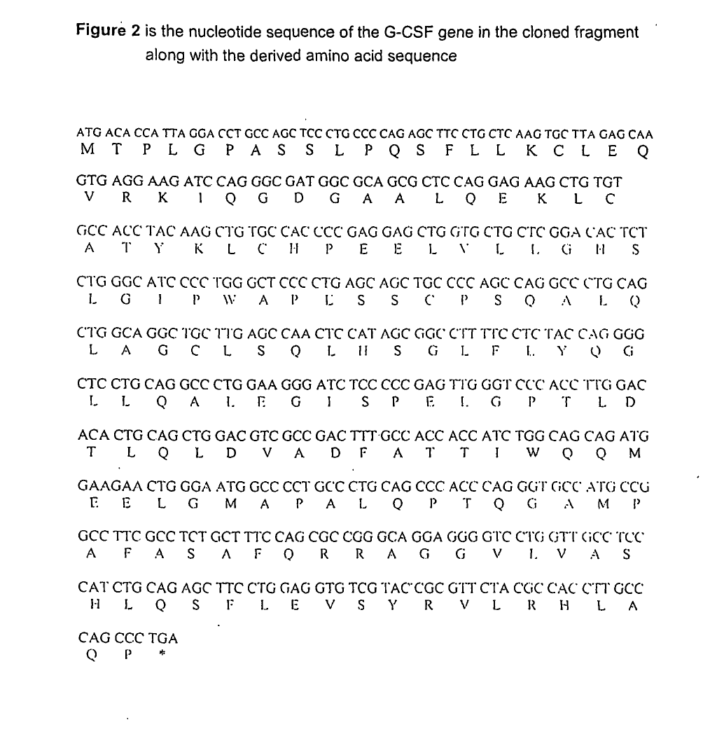

A cDNA library is constructed from a human urinary bladder carcinoma cell line. Appropriate oligonucleotide primers specific for the mature coding portion of G-CSF gene are synthesized and used to amplify the gene by RT-PCR. This is then cloned into the NdeI-EcoR I sites of the expression vector pTCF-01, suitably placed downstream of the lac-based promoter (FIG. 1). Restriction mapping and DNA sequencing is used to confirm the DNA sequence of the cloned fragment (FIG. 2). This plasmid construct is then used to transform the expression host (a strain of E. coli). The expression host harboring the plasmid construct expresses G-CSF protein at high levels when induced with IPTG or lactose. The microbial host strain used for production of recombinant G-CSF is one in which G-CSF is produced in inclusion bodies. Standard procedures as described by Sambrooket. al. (Molecular Cloning. A Laboratory Manual. Cold Spring Harbor Laboratory Press. 1989) and Pouwels et. al (Cloning Vectors: A Laboratory Manual, Elsevier, N.Y., 1985) are used in the design and use of cloning strategies and expression vectors.

### Purification

Fermentation of the recombinant E. coli strains containing the G-CSF gene is done under conditions optimized Tor maximum expression. The cells are harvested after the desired cell density is achieved and stored frozen al temperatures between −10 to −20 degrees Centigrade or processed immediately for purification.

Purification G-CSF from harvested E. coli cells is carried out by a two-step chromatography procedure of the refolded protein. The inclusion bodies containing the G-CSF protein are solubilised in 2.0-4.0 M Urea or guanidinium hydrochloride and desalted (in the case of Gdn HCl) and refolded at an acidic pH so as to be suitable for direct loading on a cation exchange column. The matrix used for cation exchange chromatography can have Carboxy Methyl, Sulpho Propyl or Sulphonate functional groups attached to resins made of cellulose, agarose or their derivatives.

The overall methodology involves lysing bacterial cells , isolating inclusion bodies and purifying the protein by ion exchange and hydrophobic chromatography. The frozen bacterial cell paste is suspended in lysis buffer, al a pellet to buffer ratio in the range of 1 gm:5 ml to 1 gm:10 ml. The lysis buffer is composed of 50 mM Tris HCl buffer, at pH8.0. 1 mM EDTA and 1 mM phenyl methyl sulfonyl fluoride (PMSF). The cell suspension is lysed by sonication or high pressure homogonization using multiple passes in the homogenizer. The cell lysate is centrifuged and the inclusion bodies are isolated from the pellet fraction.

The IB pellet is solubilized using a combination of a suitable denaturant (urea or guanidinium chloride) at alkaline pH in the range of 8.0 to 11.0. Refolding of the protein is earned out at room temperature for 6-16 hrs at acidic pH. The pH of the refolded protein solution is maintained in the range of 3.5 to 5.5 using any appropriate buffer suitable for maintaining pH in the acidic range.

A chromatography column is packed with a cation exchange matrix, which is equilibrated with a suitable buffer that can maintain the pH at an acidic range. Buffers of phosphate and acetate are preferred although citrate salts can also be used. Low ionic strengths are preferred for equilibration, with values ranging from 5 mM to 50 mM of the buffer salt and a pH range of 3.5 to 5.5. The refolded protein solution in the pH range of 3.5 to 5.5 is loaded on an ion exchange column and washed with equilibration buffer till the optical density value at 280 nm returns to baseline G-CSF is eluted from this column using a gradient of an ionic salt like chloride, citrate or sulphate in the range of 0.05 M to 0.25M. An improved recovery of G-CSF was obtained under these elution conditions and the protein was found to be homogenous with minimum amount of aggregates. The G-CSF eluate form this column is directly loaded onto a column packed with a hydrophobic matrix having butyl, oclyl or phenyl functional groups attached to a resin derived from cellulose, agarose, dextran synthetic polymers or their derivatives. The column is equilibrated at a pH below 7.0 in a suitable buffer containing 0.5 M ammonium sulphate. The bound G-CSF protein is eluted in the same buffer by gradient elution from 0.5M-0.0 M ammonium sulphate. The G-CSF protein after this step can be buffer exchanged with the final storage buffer and stored as a liquid solution at 2 to 8 degrees Centigrade without loss of activity.

### EXAMPLE 1

The following example illustrates the simplified process for solubilization of inclusion bodies and refolding of the protein at acidic pH. This example relates to the use of a combination of sub-denaturing concentrations of urea or guanidinium chloride in the concentration range of 2.0 to 4.0 M with alkaline pH for the solubilization of G-CSF from the inclusion bodies. In a preferred embodiment of this invention, 2.0M to 6.0M urea or guanidinium hydrochloride in water is added lo the IB at a ratio of 10% to 20%, wv, the pH of the solution is held constant in the range of 8.0 to 11.0 depending on the clarity of the solubilized solution, for a brief period of 15 to 30 minutes. The pH of this solution is shifted directly to an acidic pH in the range of 3.5 to 5.5 and left at room temperature for 6 to 16 hrs for refolding.

### EXAMPLE 2

This example relates to the ion exchange chromatography step that is used to purify the G-CSF protein solubilised and refolded from inclusion bodies. The refolded G-CSF is loaded onto a cation exchange column (carboxymethyl, sulphonyl or sulphopropyl functional groups) in pH range 3.5 to 5.5, preferably at pH 4.0 to 5.0 in anionic buffers that can provide buffering in this pH range for example citrate, phosphate or acetate. The buffers are generally in the molarity range of 5 mM to 50 mM preferably 10 mM to 25 mM. Washing of the column is done with the same buffer till the optical density at 280 nm comes to baseline. Elution of the protein from the column is done by a linear gradient of ionic salts containing chloride, citrate or sulphate in the concentration range of 0.0M to 0.5M in the equilibration buffer of a pH range 4.0 to 6.0. The G-CSF protein is recovered with good yields and a minimum amount of aggregated protein.

### EXAMPLE 3

This example describes the use of a hydrophobic chromatography column as a polishing step for the therapeutic grade purification of G-CSF. The cation exchange column eluate is buffer exchanged with the equilibration buffer of the hydrophobic column containing ammonium sulphate in the molarity range of 0.25 to 1.0M more preferably around 0.4 to 0.6 M. The equilibration buffer is in the acidic pH in the range of 4.0 to 7.0, more preferably in the range of 4.0 to 5.0. Elution from this column is effected by reducing the molarity of ammonium sulphate in the buffer in a continuous linear gradient elution. The protein very often clues towards the end of the gradient with improved recoveries seen when a small amount of ethanol is added to the eluting buffer preferably in the range of 2% to 20%. The hydrophobic matrix chosen can be of butyl, octyl or phenyl functional groups more preferably butyl or octyl attached to a resin derived from cellulose, agarose, dextran, synthetic polymers or their derivatives. The G-CSF protein after this step can be buffer exchanged with the final storage buffer and stored as a liquid solution at 2 to 8 degrees Centigrade without loss of activity minutes. The pH of this solution is shifted directly to an acidic pH in the range of 3.5 to 5.5 and left at room temperature for 6 to 16 hrs for refolding.

### EXAMPLE 2

This example relates to the ion exchange chromatography step that is used to purify the G-CSF protein solubilised and refolded from inclusion bodies. The refolded G-CSF is loaded onto a cation exchange column (carboxymethyl, sulphonyl or sulphopropyl functional groups) in pH range 3.5 to 5.5, preferably at pH 4.0 to 5.0 in anionic buffers that can provide buffering in this pH range for example citrate, phosphate or acetate. The buffers are generally in the molarity range of 5 mM to 50 mM preferably 10 mM to 25 mM. Washing of the column is done with the same buffer till the optical density at 280 nm comes to baseline. Elution of the protein from the column is done by a linear gradient of ionic salts containing chloride, citrate or sulphate in the concentration range of 0.0M to 0.5M in the equilibration buffer of a pH range 4.0 to 6.0. The G-CSF protein is recovered with good yields and a minimum amount of aggregated protein.

### EXAMPLE 3

This example describes the use of a hydrophobic chromatography column as a polishing step for the therapeutic grade purification of G-CSF. The cation exchange column eluate is buffer exchanged with the equilibration buffer of the hydrophobic column containing ammonium sulphate in the molarity range of 0.25 to 1.0M more preferably around 0.4 to 0.6 M. The equilibration buffer is in the acidic pH in the range of 4.0 to 7.0, more preferably in the range of 4.0 to 5.0. Elution from this column is effected by reducing the molarity of ammonium sulphate in the buffer in a continuous linear gradient elution. The protein very often elutes towards the end of the gradient with improved recoveries seen when a small amount of ethanol is added to the eluting buffer preferably in the range of 2% to 20%. The hydrophobic matrix chosen can be of butyl, oetyl or phenyl functional groups more preferably butyl oroctyl attached to a resin derived from cellulose, agarose, dextran, synthetic polymers or their derivatives. The G-CSF protein after this step can be buffer exchanged with the final storage buffer and stored as a liquid solution at 2 to 8 degrees Centigrade without loss of activity.

We claim:

1. A method for large scale purification of therapeutic grade recombinant human G-CSF, said method comprising the steps of:
isolating inclusion bodies containing G-CSF from microbial cells
solubilizing said G-CSF proteins from isolated inclusion bodies
refolding the said solubilized G-CSF proteins to obtain active refolded protein
subjecting the said refolded G-CSF protein to two step chromatography wherein the said refolded G-CSF protein is first subjected to cation exchange chromatography followed by hydrophobic interaction chromatography
to obtain purified therapeutic grade G-CSF protein

2. A method as claimed in claim 1, wherein the said G-CSF isolated from inclusion bodies is solubilized in a concentration of urea or guanidinium hydrochloride at alkaline pH and refolded at an acidic pH for 6 to 16 hrs at room temperature.

3. A method as claimed in claim 1, wherein said refolded protein is bound to a sulphonate, carboxymethyl or sulphopropyl functional group containing chromatography matrices.

4. A method as claimed in claim 1, wherein the ion exchange column is run in the pH range of 3.5 to 5.5 using buffers of citrate, phosphate or acetate salts in the molarity range of 5 mM to 50 mM.

5. A method as claimed in claim 1, wherein the said protein from the ion exchange group is eluted by increasing the ionic strength of the buffer by the addition of chloride, citrate or sulphate salts in the pH range of 4.0 to 6.0.

6. A method as claimed in claim 1 wherein the G-CSF containing protein solution eluted from the cation exchange column is purified using hydrophobic interaction chromatography on resins having butyl, oclyl or phenyl functional groups.

7. A method as claimed in claim 6, wherein the said column is equilibrated with buffers in the pH range of 4.0 to 7.0 and with ammonium sulphate salts in the molarity range of 0.25 M to 1.0 M.

8. A method as claimed in claim 6 wherein the said column is eluted by decreasing the concentration of ammonium sulphate salt in the buffer and by optionally increasing the concentration of ethanol from 2 to 20% for enhanced recoveries.
